(19) United States
(12) Patent Application Publication
(10) Pub. No.: US 2011/0296596 A1
(43) Pub. Date: Dec. 8, 2011

(54) SKIING GOGGLE FRAME WITH VENTILATION STRUCTURE

(76) Inventor: LIN-YUN CHEN, TAINAN CITY (TW)

(21) Appl. No.: 12/794,024

(22) Filed: Jun. 4, 2010

(51) Int. Cl. A61F 9/02

(52) U.S. Cl. 2/436

(57) ABSTRACT

A skiing goggle frame includes a frame having a frame periphery defining a compartment. The frame periphery includes a top portion having at least one first vent defined by a vent frame. The vent frame is inclined and extends upward from the frame periphery towards a face of a wearer wearing the skiing goggle frame. Since the head of the skier inclines forward during skiing, the vent frame and the first vent directly face the incoming wind such that the wind can directly enter a space between the lens and the face of the wearer, reducing the temperature in the space and, thus, avoiding fogging.

### BACKGROUND OF THE INVENTION

The present invention relates to a skiing goggle frame with ventilation structure and, more particularly, to a skiing goggle frame including a frame having a frame periphery that has at least one vent formed in a top portion thereof.

Skiing goggles are generally used in low-temperature environments. Due to the body heat of the wear wearing the skiing goggle, fogging is caused in a space defined between the face of the wearer and the frame of the skiing goggle and inhibits the view of the wearer, adversely affecting the skiing. U.S. Pat. Nos. 6,637,038 B1 and 7,137,153 B2 disclose sport goggles with improved ventilation by providing upper and lower intake venting apertures in upper and lower edges of the goggle frame to allow entrance of ambient air into the space between the face of the wearer and the goggle frame, lowering the temperature in the space for preventing fogging.

However, the goggle frames are designed for various sports goggles, not for skiing goggles only. Specifically, a skier generally moves in a curved path from a higher place to a lower one such that the intake venting apertures in the lower edge can not provide an efficient air intake angle for the incoming wind.

Thus, a need exists for a skiing goggle frame that obviates and/or mitigates this disadvantage.

### BRIEF SUMMARY OF THE INVENTION

The present invention solves this need and other problems in the field of effective ventilation during skiing by providing, in a preferred form, a skiing goggle frame includes a frame having a frame periphery defining a compartment. The frame periphery includes a top portion having at least one first vent defined by a vent frame. The vent frame is inclined and extends upward from the frame periphery towards a face of a wearer wearing the skiing goggle frame.

A lens can be mounted to the frame periphery of the frame and align with the compartment to form a skiing goggle. A wearing portion can be attached to the frame periphery and can be worn by the wearer. Since the head of the skier inclines forward during skiing, the vent frame and the first vent directly face the incoming wind such that the wind can directly enter a space between the lens and the face of the wearer, reducing the temperature in the space and, thus, avoiding fogging.

The present invention will become clearer in light of the following detailed description of illustrative embodiments of this invention described in connection with the drawings.

### DESCRIPTION OF THE DRAWINGS

The illustrative embodiments may best be described by reference to the accompanying drawings where:

FIG. 1 shows a perspective view of a skiing goggle frame according to the preferred teachings of the present invention.

FIG. 2 shows a top view of the skiing goggle frame of FIG. 1.

FIG. 3 shows a top view of the skiing goggle frame of FIG. 1 with filters mounted in the skiing goggle frame.

FIG. 4 shows a perspective view of the skiing goggle frame of FIG. 3, illustrating ventilation effect provided by the skiing goggle frame.

FIG. 5 shows a perspective view illustrating the skiing goggle frame of FIG. 1 worn on a face of a wearer.

FIG. 5A shows an enlarged, cross sectional view of a circled portion of FIG. 5, illustrating the ventilation effect.

FIG. 6 shows an enlarged view of a circled portion of FIG. 1, illustrating entrance of air through a guide hole in a side of the skiing goggle frame.

FIG. 7 shows a partial, side view of the skiing goggle frame of FIG. 1, illustrating exit of air through air outlets in the side of the skiing goggle frame.

All figures are drawn for ease of explanation of the basic teachings of the present invention only; the extensions of the figures with respect to number, position, relationship, and dimensions of the parts to form the preferred embodiments will be explained or will be within the skill of the art after the following teachings of the present invention have been read and understood. Further, the exact dimensions and dimensional proportions to conform to specific force, weight, strength, and similar requirements will likewise be within the skill of the art after the following teachings of the present invention have been read and understood.

### DETAILED DESCRIPTION OF THE INVENTION

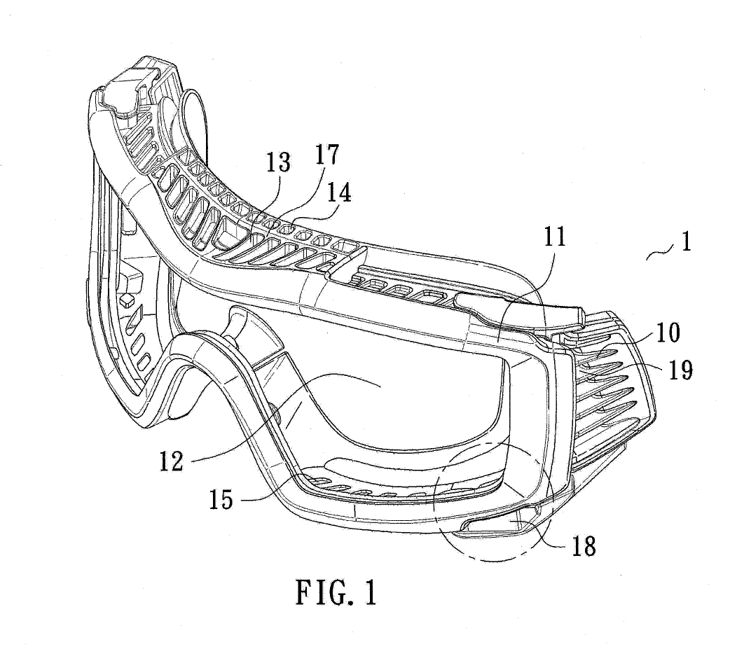

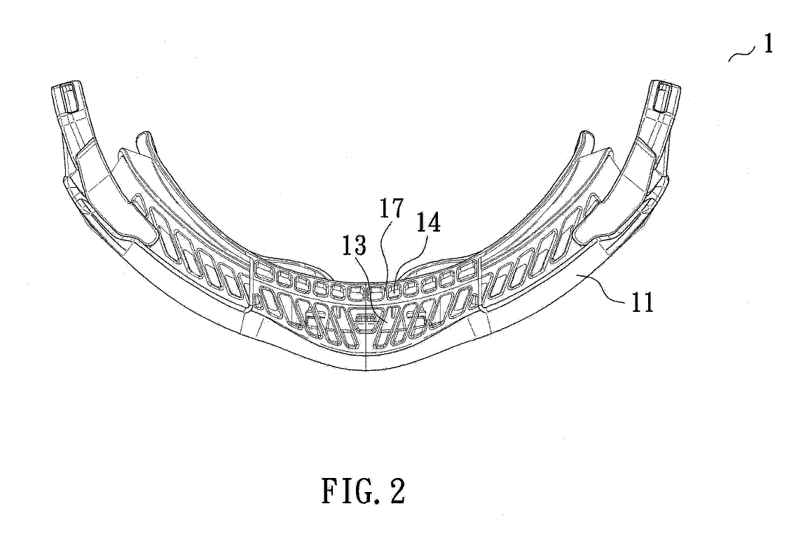

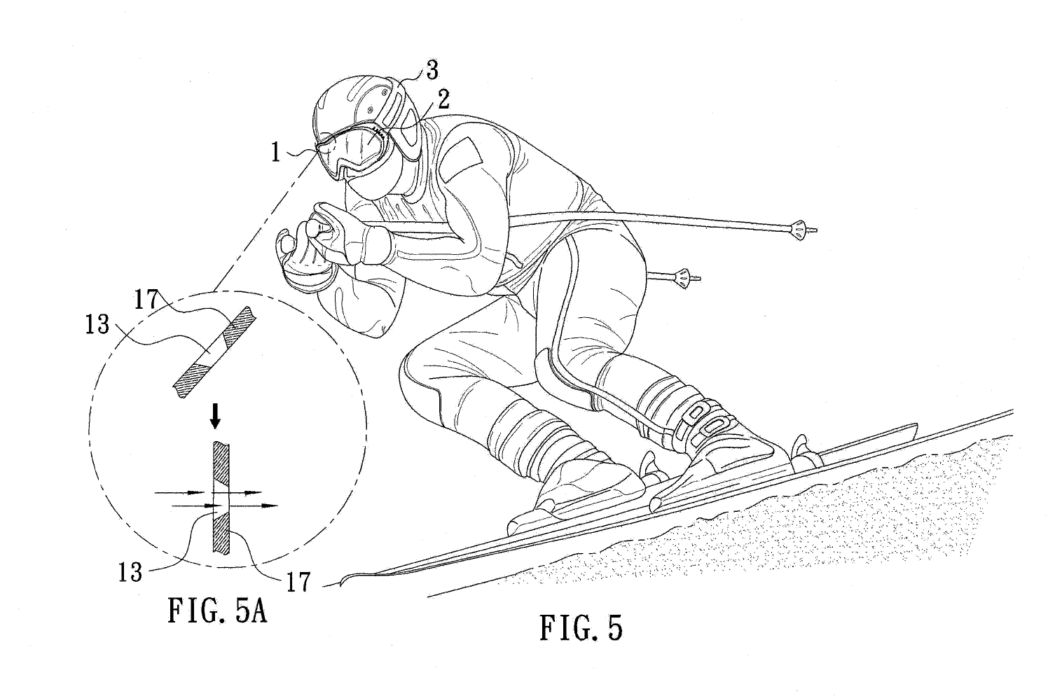

With reference to FIGS. 1-3, a skiing goggle frame according to the preferred teachings of the present invention generally includes a frame 1 having a frame periphery 11 defining a compartment 12. At least one first vent 13 is formed in a top portion of the frame periphery 11. The first vent 13 is defined by a vent frame 17. The vent frame 17 is inclined and extends upward from the frame periphery 11 towards a face of a wearer wearing the skiing goggle frame. A lens 2 is mounted to the frame periphery 11 of the frame 1 and aligned with the compartment 12 to form a skiing goggle, as shown in FIG. 5. A wearing portion 3 can be attached to the frame periphery 11 and can be worn by the wearer.

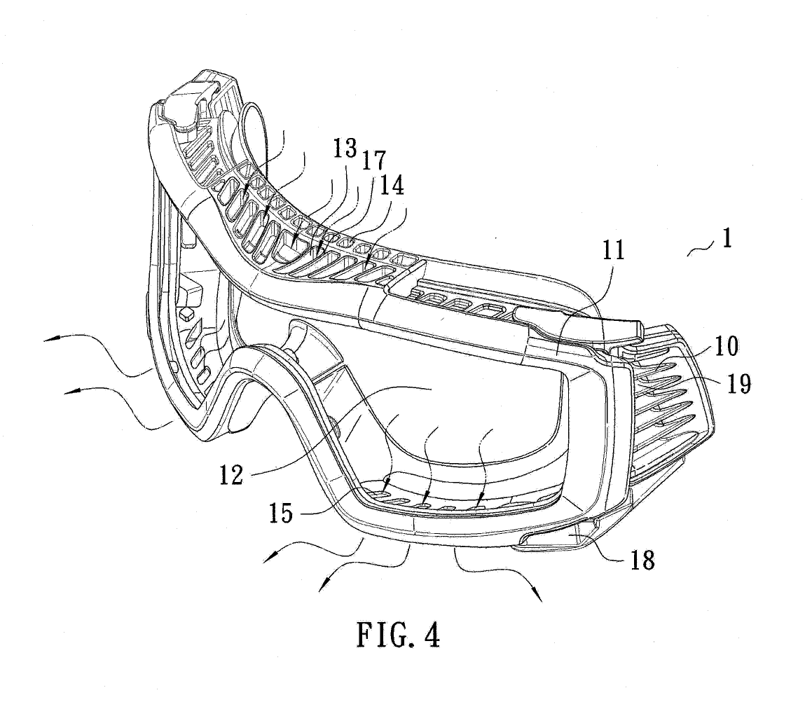

With reference to FIGS. 1 and 4, the skiing goggle frame according to the preferred teachings of the present invention can further include a second vent 14 (or more second vents 14) in the top portion of the frame periphery 11 of the frame 1 and adjacent to the first vent 13 to provide more amount of air.

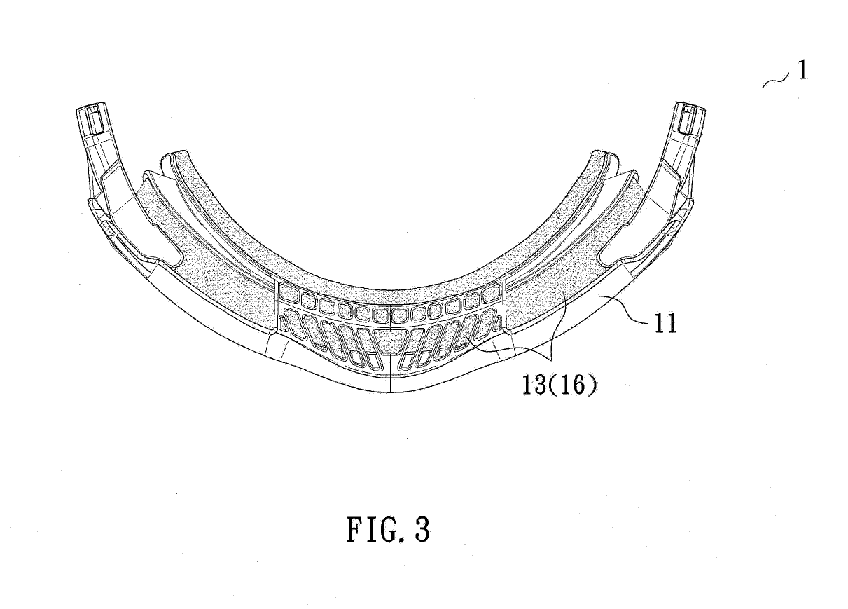

With reference to FIG. 3, the skiing goggle frame according to the preferred teachings of the present invention can include a filter 16 mounted to the top portion of the frame periphery 11 of the frame 1 and covering a bottom edge of the first vent 13. It can be appreciated that the filter 16 can be mounted to cover a top edge of the first vent 13.

To enhance the convection of air, at least one vent 15 can be formed in a bottom portion of the frame periphery 11 of the frame 1, as shown in FIG. 1.

With reference to FIG. 5, since the head of the skier (the wearer) inclines forward during skiing, the vent frame 17 and the first vent 13 directly face the incoming wind such that the wind can directly enter a space between the lens 2 (FIG. 5) and the face of the wearer, reducing the temperature in the space and, thus, avoiding fogging.

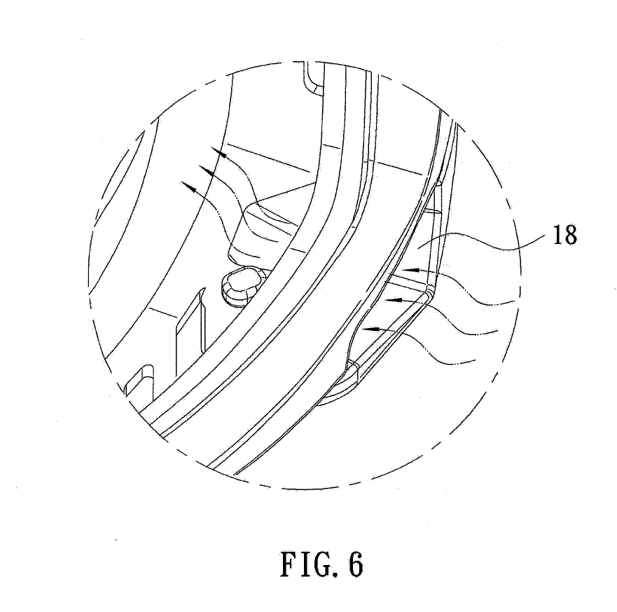

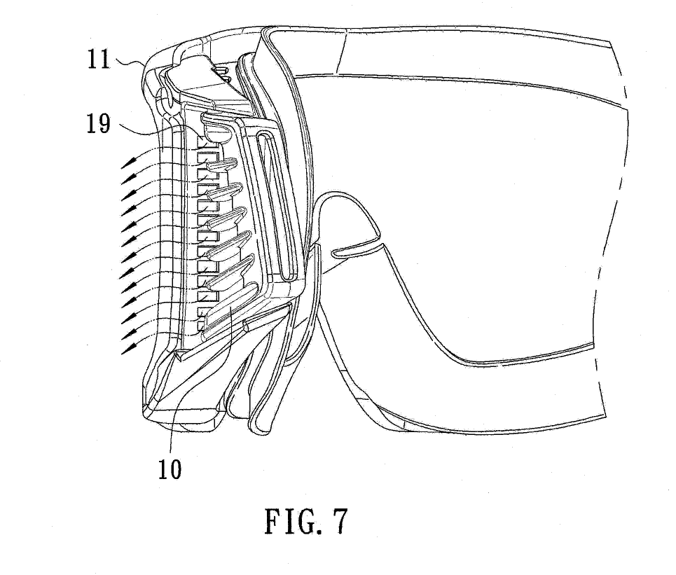

With reference to FIGS. 1, 4, 6, and 7, the frame periphery 11 of the skiing goggle frame according to the preferred teachings of the present invention further includes a guide hole 18 formed in a bottom edge thereof adjacent the outer portion and in communication with the space between the lens 2 and the face of the wearer. Furthermore, at least one air outlet 19 is formed in an outer portion of the frame periphery 11. In the most preferred form shown, the frame periphery 11 includes a plurality of air outlets 19 defined by a plurality of partitioning boards 10 and in communication with the space between the lens 2 and the face of the wearer. Thus, air guided into the space between the lens 2 and the face of the wearer via the guide hole 18 can exit the frame 1 via the air outlets 19 in the outer portion of the frame periphery 11. The heat generated by the wearer is, thus, exhausted at increased speed.

Thus since the invention disclosed herein may be embodied in other specific forms without departing from the spirit or general characteristics thereof, some of which forms have been indicated, the embodiments described herein are to be considered in all respects illustrative and not restrictive. The scope of the invention is to be indicated by the appended claims, rather than by the foregoing description, and all changes which come within the meaning and range of equivalency of the claims are intended to be embraced therein.

1. A skiing goggle frame comprising a frame having a frame periphery defining a compartment, with the frame periphery including a top portion having at least one first vent defined by a vent frame, with the vent frame being inclined and extending upward from the frame periphery towards a face of a wearer wearing the skiing goggle frame.

2. The skiing goggle frame as claimed in claim 1, with the frame further including a second vent in the top portion of the frame periphery and adjacent to said at least one first vent.

3. The skiing goggle frame as claimed in claim 1, further comprising: a filter mounted to the top portion of the frame periphery of the frame and covering a bottom edge of said at least one first vent.

4. The skiing goggle frame as claimed in claim 1, with the frame periphery of the frame including a bottom portion having at least one second vent.

5. The skiing goggle frame as claimed in claim 1, with the frame periphery including an outer portion and a bottom edge adjacent the outer portion, with the bottom edge including a guide hole in communication with the space between the lens and the face of the wearer, with at least one air outlet formed in the outer portion of the frame periphery and in communication with the space between the lens and the face of the wearer.
